SEQUENCE LISTING (2 sequences)

SEQ ID NO: 1 - protein, 207 aa, Homo sapiens
  human FGF-18
MYSAPSACTCLCLHFLLLCFQVQVLVAEENVDFRIHVENQTRARDDVSRKQLRLYQLYSRTSGKHIQVLGRRISARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQPELQKPFKYTTVTKRSRRIRPTHPA

SEQ ID NO: 2 - protein, 170 aa, Artificial
  Recombinant truncated FGF-18(trFGF-18)
MEENVDFRIHVENQTRARDDVSRKQLRLYQLYSRTSGKHIQVLGRRISARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQPELQKPFKYTTVTK

(19) United States
(12) Patent Application Publication
(10) Pub. No.: US 2020/0128812 A1
(43) Pub. Date: Apr. 30, 2020

(54) CARTILAGE PRESERVATION MEDIA

(71) Applicants: MERCK PATENT GMBH, DARMSTADT (DE); THE TRUSTEES OF THE UNIVERSITY OF PENNSYLVANIA, PHILADELPHIA, PA (US)

(72) Inventors: ANNE GIGOUT, GRIESHEIM (DE); HANS GUEHRING, GEISENHEIM (DE); GEORGE R. DODGE, PHILADELPHIA, PA (US); ROBERT L. MAUCK, PHILADELPHIA, PA (US)

(21) Appl. No.: 16/494,407

(22) PCT Filed: Mar. 17, 2017
(86) PCT No.: PCT/EP2017/056448

§ 371 Date: Sep. 16, 2019

(51) Int. Cl. A01N 1/02; C12N 5/077

(52) U.S. Cl. CPC A01N 1/0226; C12N 2501/119; C12N 5/0655

(57) ABSTRACT

The invention relates to the field of storage of tissues, such as transplantable material, or allograft storage, and more specifically to the field of both short- and long-term tissues storage and preservation. Preferably the tissues are transplantable material comprises chondogenic cells, such as chondrocytes, cartilage, engineered cartilage or to osteochondral/cartilage explants or fragment thereof.

### FIELD OF THE INVENTION

The invention relates to the field of storage of tissues, such as transplantable material, or allograft storage, and more specifically to the field of both short- and long-term tissue storage and preservation. Preferably the tissue is a transplantable material comprising chondogenic cells, such as chondrocytes, cartilage, engineered cartilage or to osteochondral/cartilage explants or fragment thereof.

### BACKGROUND OF THE INVENTION

Allograft or other transplantable material can be used to treat many diseases and/or defects, such as arthritis or osteoarthritis. These grafts, originating from organ donors may have to be stored before transplantation into patients. For instance, osteochondral or cartilage allografts are currently preserved at 4° C. and used within 28 days of donor harvest. The storage period can be also needed simply for viral and other safety testing. In the case of osteochondral or cartilage allografts, the safety period is very often of about 2 weeks, leading to a window for transplantation of only 14 days. Storage conditions for allograft or other tissue samples may influence tissue viability, integrity, and/or sterility. Indeed, in vitro storage or periods of culture of cartilage results in changes in cartilage characteristics and mechanical function that is likely due to the loss or degradation of extracellular matrix (ECM) as well as chondrocyte viability ([1,2]). Cartilage has complex mechanical properties including biphasic behavior ([3]), tension-compression non-linearity ([3]), and depth-dependent properties ([4]). In degenerative diseases, the ECM is disrupted and mechanical function becomes more isotropic, similar to what is seen during in vitro periods.

Attempts to preserve the native cartilage phenotype during in vitro culture have been met with varying degrees of success.

There is a need for improved tissues storage conditions, either via the use of improved media enhancing the viability and/or stability of the tissues over time (e.g. helping to preserve the native mechanical properties and native inhomogeneity of cartilage), or of improved methods of storage.

### SUMMARY OF THE INVENTION

One aspect of the invention provides a method for short term storage of tissues, such as transplantable material, comprising the step of incubating (or maintaining) a tissue's composition, while maintaining its mechanical properties and cellular viability in a storage medium, preferably at a temperature of about 20 to about 38° C. for up to 5 weeks, until use in surgery, or any transplantation methods such as grafts. In another aspect of the invention is described a method for long term storage of tissues, such as transplantable material, comprising the step of incubating (or maintaining) a tissue in a storage medium, preferably at a temperature of about 20° C. to about 38° C., for at least 5 weeks, until use in surgery, or any transplantation methods such as grafts.

A further aspect of the invention is a medium for storage of transplantable material that comprises an FGF-18 compound, salts, amino acids, at least one preservative (such as at least one antibiotic compound and/or at least one antimycotic compound), vitamins, a buffer and optionally a serum or a serum component. Said medium is preferably free of lipids. It is also preferably free of compounds such as dexamethasone. Said medium is called storage medium.

The invention also provides methods of making a storage medium comprising FGF-18, comprising the step of combining the ingredients to make the medium.

The tissue to be stored according to the invention is preferably of cartilage or osteochondral origin. Said tissue can be originating from, meniscus, scapula, humerus, radius, ulna, femur, tibia), patella, talus, phalanges or temporomandibular joint tissue.

In another aspect, the invention provides kits for preserving (or storing) tissue including the storage medium according to the invention, a vial for storage and instructions for use according to the invention.

### DESCRIPTION OF THE DRAWINGS

FIG. 1: Equilibrium modulus of cartilage explants normalized to week 0 (*p<0.05 vs week 0, #=p<0.05 vs control) n=18-23 controls, n=17-29 treated).

FIG. 2: Equilibrium modulus as a function of depth from the surface of the cartilage explants. Week 0 baseline modulus is shown in light grey, control as dashed lines, and sprifermin treated samples as dark grey lines. Error bars indicate standard error of the mean.

FIG. 3: (A) Collagen content normalized to wet weight. (B) GAG Content normalized to wet weight. (C) Weekly matrix metalloproteinase (MMP) concentration in the medium (*=p<0.05 vs week 0, #=p<0.05 vs control).

### DEFINITION

The term “FGF-18 compound” or “FGF-18”, as used herein, is intended to be a protein maintaining at least one biological activity of the human FGF-18 protein. FGF-18 may be native, in its mature form, or a truncated form thereof. Biological activities of the human FGF-18 protein include notably the increase in osteoblastic activity ([5]) or in cartilage formation ([6]). Native, or wild-type, human FGF-18 is a protein expressed by chondrocytes of articular cartilage. Human FGF-18 was first designated zFGF-5 and is fully described in ([5]). SEQ ID NO:1 corresponds to the amino acid sequence of the native human FGF-18, with a signal peptide consisting of amino acid residues 1(Met) to 27(Ala). The mature form of human FGF-18 corresponds to the amino acid sequence from residue 28(Glu) to residue 207(Ala) of SEQ ID NO: 1 (180 amino acids). The term also includes fusion protein, wherein FGF-18 protein is coupled with a heterologous protein or a chemical compound.

FGF-18, in the present invention, may be produced by recombinant methods, such as taught by the application ([7]). Depending on the expression systems and conditions, FGF-18 in the present invention is expressed in a recombinant host cell with a starting Methionine (Met) residue or with a signal sequence for secretion. When expressed in prokaryotic host, such as in E. coli, FGF-18 contains an additional Met residue in N-terminal of its sequence. For instance, the amino acid sequence of human FGF-18, when expressed in E. coli, starts with a Met residue in N-term (position 1) followed by residues 28 (Glu) to residue 207 (Ala) of SEQ ID NO: 1.

The term “truncated form” of FGF-18, as used herein, refers to a protein which comprises or consists of residues 28(Glu) to 196(Lys) of SEQ ID NO: 1. Preferably, the truncated form of FGF-18 protein is the polypeptide designated “trFGF-18” (170 amino acids), which starts with a Met residue (in N-terminal) followed by amino acid residues 28 (Glu)-196 (Lys) of the wild-type human FGF-18. The amino acid sequence of trFGF-18 is shown in SEQ ID NO:2 (amino acid residues 2 to 170 of SEQ ID NO:2 correspond to amino acid residues 28 to 196 of SEQ ID NO:1). trFGF-18 is a recombinant truncated form of human FGF-18, produced in E. coli ([7]). The International Nonproprietary Name (INN) for this particular form of FGF-18 is sprifermin. Sprifermin has been shown to display similar activities as the mature human FGF-18, e.g. it increases chondrocyte proliferation and cartilage deposition leading to repair and reconstruction for a variety of cartilaginous tissues ([6]).

“Cartilage disorder”, as used herein, encompasses disorders resulting from damages due to injury, such as traumatic injury, chondropathy or arthritis. Examples of cartilage disorders that may be treated by the administration of the FGF-18 formulation described herein include but are not restricted to arthritis, such as osteoarthritis, cartilage injury, fractures affecting joint cartilage or surgical procedures with impact on joint cartilage (e.g. Microfracture). Degenerative diseases/disorders of the cartilage or of the joint, such as chondrocalcinosis, polychondritis, relapsing polychondritis, ankylosing spondylitis or costochondritis are also encompassed by this wording. The International Cartilage Repair Society has proposed an arthroscopic grading system to assess the severity of the cartilage defect: grade 0: (normal) healthy cartilage, grade 1: the cartilage has a soft spot or blisters, grade 2: minor tears visible in the cartilage, grade 3: lesions have deep crevices (more than 50% of cartilage layer) and grade 4: the cartilage tear exposes the underlying (subchronal) bone (see for instance page 13 of http://www.cartilage.org/_files/contentmanagement/ICRS_evaluation.pdf).

The term “Osteoarthritis” is used to intend the most common form of arthritis. The term “osteoarthritis” encompasses both primary osteoarthritis and secondary osteoarthritis (see for instance The Merck Manual, 17^{th} edition, page 449). The most common way of classifying/grading osteoarthritis is the use of the Kellgren-Lawrence radiographic grading scale (see table below). Osteoarthritis may be caused by the breakdown of cartilage. Bits of cartilage may break off and cause pain and swelling in the joint between bones. Over time, the cartilage may wear away entirely, and the bones will rub together. Osteoarthritis can affect any joint but usually concerns hands and weight-bearing joints such as hips, knees, feet, and spine. In a preferred example, the osteoarthritis may be knee osteoarthritis or hip osteoarthritis.

The term “cartilage injury” as used herein is a cartilage disorder or cartilage damage resulting notably from a trauma. Cartilage injuries can occur notably after traumatic mechanical destruction, notably further to an accident or surgery (for instance microfracture surgery). This term “cartilage injury” also includes chondral or osteochondral fracture, damage to meniscus, and the term microfracture. Also considered within this definition is sport-related injury or sport-related wear of tissues of the joint.

The term “tissue engineering” encompasses also autologous chondrocyte implantation (ACI). It is also known as regenerative medicine. Cells or tissues can be cultivated either in monolayer cultures or in 3D cultures. The aim of such procedures is to repair or replace parts of or whole tissues.

The term “graft” is related to transplantation or implantation. This procedure is also part of regenerative medicine. It includes osteochondral or cartilage (also referred to herein as osteochondral/cartilage) transplantation/implantation, such as engineered cartilage, osteochondral/cartilage autograft or osteochondral/cartilage allograft transplantation/implantation. In the frame of a graft, an explants is harvest from a mammal, either from the mammal to be treated (i.e. autograft) or from another mammal preferably of the same species (allograft). Usually, it is taken from a health cartilage section or from a healthy ostheochondral tissue.. Such graft is preferably performed at the level of the cartilage defect(s).

The term “tissue” or “tissues” herein refer to connective tissues and more particularly to cartilage connective tissue. Preferably it is a transplantable material according to the invention.

The term “transplantable cartilage material” or “transplantable material” are used interchangeably. They refer to chondogenic cells, such as chondrocytes, engineered cartilage or osteochondral/cartilage explants that are prepared in order to be transplanted (or implanted) in a mammal in need thereof. Such transplantable material is preferably to be transplanted/implanted at the level of the cartilage defect(s). Alternatively it can be used for research purpose. Said transplantable material can be removed from a donor by routine techniques. The donor can be a living subject or a cadaver. Said donor can originating from all types of mammaly, including, but not limited to human, porcine, ovine, bovine, canine, equine, and others.

### DETAILED DESCRIPTION

There is a need for improved storage conditions, either via the use of improved media enhancing the viability and/or stability of tissues over time or of improved methods of tissues storage. Preferably, the tissue is a transplantable material or allorgraft material. Said tissue can be stored for instance before surgery, graft or research. The inventors have found a improved method of storage enhancing viability and/or stability of tissues, such as transplantable material, until use in surgery, research, or any transplantation methods such as grafts. It has been surprisingly found that using said method, the tissues can be stored for at least 4 weeks at a temperature of about 20° C. to about 38° C., while keeping both viability, stability and mechanical properties of said tissues. They have also identified an improved medium to be used in methods for storing tissues until use in surgery, research, or any transplantation methods such as grafts.

In a first embodiment, herein described is a method for short term storage (storage for up to 5 weeks) of tissues, such as transplantable material. It was indeed surprisingly shown by the inventors that both viability and stability were preserved for up to 5 weeks at 37° C. when using the methods according to the invention. Therefore herein disclosed is a method for short term storage of tissues, transplantable material, comprising the step of incubating (or maintaining) the transplantation material in a storage medium at a temperature of about 20° C. to about 38° C., for up to 5 weeks, wherein said storage medium comprises FGF-18.

It has been found that storage of tissue (e.g. transplantable material) may be facilitated by replacement of old culture storage medium with fresh medium. The storage medium can be conveniently changed as necessary. In one embodiment, the medium is changed at least once, twice, or three times during storage. Alternatively the medium can be changed every week until the transplantatble material is used.

In an alternative embodiment, the tissues can be stored at a temperature of about 20° C. to about 38° C. for up to 5 weeks according to the following method:
1) About 24 hours (i.e. about 1 day) in fresh storage medium comprising FGF-18,
2) About 144 hours (i.e. about 6 days) in fresh storage medium not comprising FGF-18,
3) Repetition of steps 1) and 2) until end of storage.

Preferably, the storage media used in steps 1) and 2) are identical except for what concerns FGF-18.

In another embodiment, a method for long term storage (more than 5 weeks) of tissues, such as transplantable material, is described. More particularly herein described is a method for long term storage of tissues, such as transplantable material, comprising the step of incubating (or maintaining) said tissues in a storage medium at a temperature of about 20° C. to about 38° C. for at least 5 weeks, at least 6 weeks, at least 7 weeks or even more, wherein said storage medium comprises FGF-18.

It has been found that long-term storage of tissue (e.g. transplantable material) may be facilitated by replacement of old culture storage medium with fresh medium. The storage medium can be conveniently changed as necessary. In one embodiment, the medium is changed at least once, twice, or three times during storage. Alternatively the medium can be changed every week until the transplantatble material is used.

In an alternative embodiment, the tissues can be stored at a temperature of about 20° C. to about 38° C. for at least 5 weeks, at least 6 weeks, at least 7 weeks or even more, according to the following method:
1) About 24 hours (i.e. about 1 day) in fresh storage medium comprising FGF-18,
2) About 144 hours (i.e. about 6 days) in fresh storage medium not comprising FGF-18,
3) Repetition of steps 1) and 2) until end of storage.

Preferably, the storage media used in steps 1) and 2) are identical except for what concerns FGF-18. In the context of the invention as a whole, the tissues is preferably a transplantation material of cartilage or osteochondral origin. Such transplantable material is preferably to be transplanted/implanted at the level of the cartilage defect(s). It can also be used for research purpose. Said tissues can be removed from a donor by routine techniques. The donor can be a living subject or a cadaver. Said donor can originating from all types of mammaly including, but not limited to human, porcine, ovine, bovine, canine, equine, and others. When used in surgery or in any transplantation methods such as grafts, the tissue is preferably from the same species as the one of the subject to be treated (e.g human origin for a human subject to be transplanted). The tissues to be stored prior to surgery or any kind of transplantations (including graft technics) can be used for treating a subject having cartilage disorders such as osteoarthritis and cartilage injury.

In the context of the invention as a whole, for both short- and long-storages, the tissues can be stored at a temperature of about 20° C. to about 38° C., preferably of about 23° C. to about 38° C., such as at or about any one of 23° C., 24° C., 25° C., 26° C., 27° C., 28° C., 29° C., 30° C., 31° C., 32° C., 33° C., 34° C., 35° C., 36° C., 37° C. or 38° C.

It was shown by the inventors that an efficient storage medium did not need to comprise all the basic components of a culture medium; it is indeed preferably free of lipid components or other usual compounds such as dexamethasone. The storage medium of the invention comprises salts, free amino acids, at least one preservative (such as at least one antibiotic compound and/or at least one antimycotic compound), at least one vitamin, a buffer and optionally a serum, such as a defined serum or a serum component. The storage medium further comprises FGF-18 either from the begining or as a supplement added extemporaneously.

According to the invention as a whole the FGF-18 (or FGF-18 compound) is preferably selected from the group consisting of 1) a polypeptide comprising or consisting of the amino acid residues 28-207 of SEQ ID NO:1, 2) a polypeptide comprising or consisting of the amino acid residues 28-196 of SEQ ID NO:1, and 3) a polypeptide comprising or consisting of SEQ ID NO:2. Its final concentration in the storage medium is from about 10 to about 300 ng/mL, preferably from about 50 to about 200 ng/mL, more preferable from about 75 to about 150 ng/mL, even more preferably from about 90 to about 110 ng/mL, such as about 90, 95, 96, 97, 98, 99, 100, 101, 102, 103, 104, 105 or 110 ng/mL.

According to the invention as a whole, the at least one preservative is at least one antibiotic compound such as is penicillin and/or streptomycin and/or at least one antimycotic compound. Preferably, if at least one antibiotic compound is used, it is present in the storage medium. Each of the antibiotic compound is in a final concentration in the storage medium of about 10 to about 300 U/mL, preferably from about 50 to about 200 U/mL, more preferable from about 75 to about 150 U/mL, even more preferably from about 90 to about 100 U/mL, such as about 90, 95, 100, 105 or 110 U/mL. Preferably, if at least one antimycotic compound is used, it is present in the storage medium. It can be, without any limitation, fungizone or gentamycin. Its final concentration in the storage medium is from about 1 to about 10 μg/mL, preferably from about 1.5 to about 5 μg/mL, more preferably from about 2 to about 3 μg/mL, such as 2.0, 2.1, 2.2, 2.3, 2.4, 2.5, 2.6, 2.7, 2.8, 2.9 or 2.9 μg/mL.

According to the invention as a whole, the at least one vitamines is MEM vitamins and/or ascorbic acid. Preferably both types of vitamins are present. MEM vitamins is used at a concentration of 1×. The ascorbic acid concentration, when added, is at a final concentration from about 10 to about 100 μg/mL, preferably from about 20 to about 80 μg/mL, more preferably from about 40 to about 60 μg/mL, such as 40, 42.5, 45, 47.5, 50, 52.5, 55, 57.5 or 60 μg/mL.

The buffer according to the invention is preferably HEPES buffer, or HEPES/sodium bicarbonate, keeping the medium at a pH between 7.2-7.4. Its final concentration is preferably at a final concentration from about 10 to 50 mM, more preferably from about 15 to about 35 mM, even more rpeferably from about 20 to 30 mM, such as 20, 21, 22, 23, 24, 25, 26, 27, 28, 29 or 30 mM.

In the context of the invention as a whole the tissues storage medium can further comprise a serum, such as a defined serum (e.g. fetal bovine serum) or a serum component (such as TGFβ). Common concentrations for said serum will be used. For instance, when said medium comprises fetal bovine serum, its concentration can be from 5 to 15%, preferably 8 to 12, such as 8, 9, 10, 11 or 12%.

The salts and amino acids according to to invention can be brought separately or can be found in commercial media such as, without any limitation, Minimum Essential Media (MEM), Dullbecco's Modified Eagle Medium (DMEM) high or low glucose, Ham's F12 or Roswell Park Memorial Institute medium (RPMI) 1640 .

Although not limitating, an example of storage medium is a medium consisting of FGF-18, DMEM 4.5 g/L D-Glucose and L-Glutamine, 10% FBS, 100 U/mL penicillin,100 μg/mL streptomycin, 2.5 μg/mL fungizone, 1% MEM vitamins, 25 mM HEPES, and 50 μg/ml vitamin C. An other example is , DMEM 4.5 g/L D-Glucose and L-Glutamine, 10% FBS, 100 U/mL penicillin,100 μg/mL streptomycin, 2.5 μg/mL fungizone, 1% MEM vitamins, 25 mM HEPES, and 50 μg/ml vitamin C to which FGF-18 can be added at any time before use, such as at the time of preparation or extemporaneously.

Herein described is a method for making the storage medium according to the invention, comprising the step of combining the ingredients to make said medium, i.e. by combining FGF-18, salts, free amino acids, at least one preservative, at least one vitamin, a buffer and optionally a serum (such as a defined serum or a serum component). Alternatively, the FGF-18 compound can be added extemporaneously to the storage medium, which in such a case will be prepared as above except for FGF-18.

Also described herein is a kit for preserving tissues including the storage medium or media according to the invention, a vial for storage and instructions for use. Said kit can contain both storage medium without FGF-18 and storage medium with FGF-18. Alternatively it can contain only storage medium without FGF-18, separate FGF-18 and instructions to prepare extemporaneously the storage medium by supplementing it with FGF-18.

The storage medium and the method according to the invention provides for preservation of at least 70% of the tissues after storage at a temperature of about 20° C. to about 38° C. for at least 30 days. In an embodiment, at least 60% or 70%, up to at least around 99%, including 60%, 65%, 70%, 75%, 80%, 85%, 90%, or 95% or greater of the cell and tissue viability and composition and mechanical properties is preserved when stored for up to 45 days, 60 days, or 70 days.

### DESCRIPTION OF THE SEQUENCES

SEQ ID NO.1: Amino acid sequence of the native human FGF-18.

SEQ ID NO.2: Amino acid sequence of the recombinant truncated FGF-18 (trFGF-18).

### EXAMPLES

Material and Methods

Explant Preparation

Full-thickness articular cartilage explants were harvested aseptically from juvenile bovine stifle joints (age 3-6 months) with a biopsy punch (4 mm diameter). Explants were cultured overnight in complete medium (DMEM 4.5 g/L D-Glucose and L-Glutamine, 10% FBS, 100 U/mL penicillin,100 μg/mL streptomycin, 2.5 μg/mL fungizone, 1% MEM vitamins (Gibco #11120), 25 mM HEPES, and 50 μg/ml vitamin C).

Following overnight culture, explants were cultured for up to 6 weeks in complete medium only (control) or complete medium with weekly 24 hour exposure to 100 ng/ml of sprifermin (Merck Serono KGaA, Darmstadt, Germany).

Mechanical Testing

To determine the local mechanical properties of the explants, samples were halved and stained with Hoechst 33342 before being imaged on a custom inverted microscope mounted loading device, and processed in Vic2D and MATLAB using cell nuclei as fiducial markers.

The bulk modulus was determined by averaging the tracked strain of the tissue from 10% to 90% of the depth. Additional samples were left intact and a 10% strain was applied using a custom device and the equilibrium load was recorded.

Biochemical Analysis

Following mechanical testing, samples were digested with Proteinase K. Collagen and glycosaminoglycan (GAG) content were quantified with orthohydroxyproline (OHP) and dimethylmethylene blue (DMMB) assays, respectively. The change in water content was also calculated. Throughout explant culture, medium was collected and the matrix metalloproteinase (MMP) concentration in the media was determined using the AnaSpec assay (AnaSpec, San Jose, Calif., USA).

Results

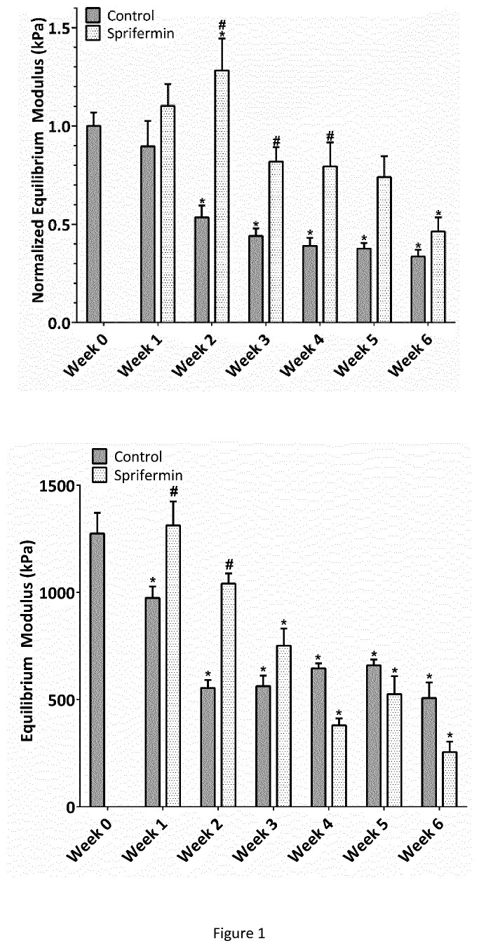

Bulk Mechanical Properties (FIG. 1)

Under control conditions, the bulk mechanical properties of explants decreased after 2 weeks in culture. Weekly sprifermin treatment maintained bulk mechanical properties for up to 5 weeks in culture. A significant decrease in equilibrium modulus was observed at week 6 compared to week 0 controls.

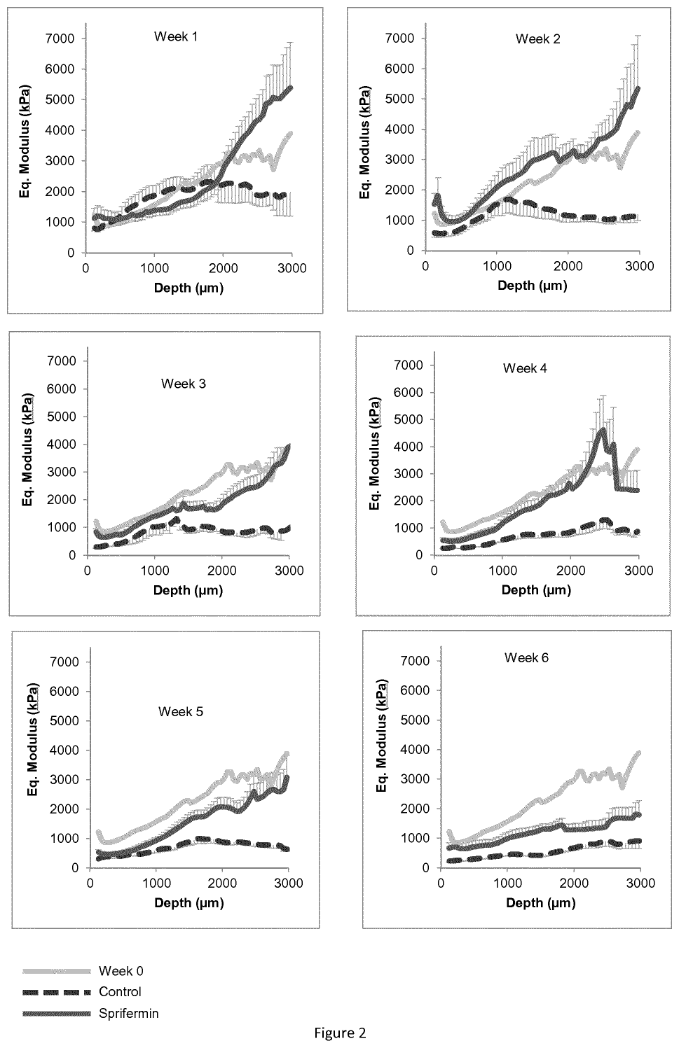

Local Mechanical Properties (FIG. 2)

When examining the depth-dependent moduli, the control samples had a decreased modulus at all time points further from the cartilage surface. Sprifermin treated samples had an increased modulus in the deeper zones at week 1 and maintained these depth-dependent mechanical properties through week 5.

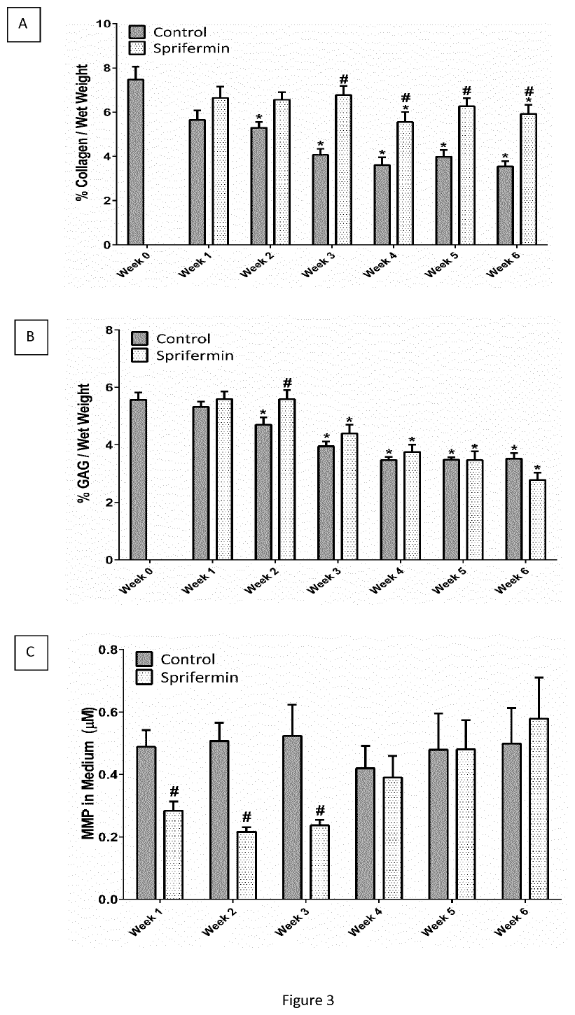

Biochemical Analysis (FIG. 3)

GAG content decreased with culture duration in both groups, while collagen content was preserved with sprifermin treatment vs. control. During the same time course, there was a significant suppression of active MMP in the media during the first 3 weeks of culture, before returning to control levels.

Conclusion

The data presented herein demonstrate the ability of FGF-18 compound, such as sprifermin, to maintain the depth-dependent properties of cartilage in method for short-term (up to 5 weeks) long-term (more than 5 weeks) in vitro culture or storage of transplantable material.

It was herein shown that FGF-18 compound, such as sprifmerin, has the potential to improve current transplantation practice by maintaining native mechanical properties of cartilage during tissue storage (for both short-term or long-term storage) prior to surgery. This represents a notable clinical and translationally-relevant finding.

### REFERENCES

[1] Acosta, Carlos A., et al. “Cell viability and protein composition in cryopreserved cartilage.” Clinical orthopaedics and related research 460 (2007): 234-239;

[2] Malinin, Theodore, H. Thomas Temple, and Bill E. Buck. “Transplantation of osteochondral allografts after cold storage.” The Journal of Bone & Joint Surgery 88.4 (2006): 762-770;

[3] Mow, Van C., et al. “Biphasic creep and stress relaxation of articular cartilage in compression: theory and experiments.” J Biomech Eng 102.1 (1980): 73-84;

[4] Soltz, M. A., and Ateshian, G. A., 1998, “Experimental Verification, and Theoretical Prediction of Cartilage Interstitial Fluid Pressurization at an Impermeable Contact Interface in Confined Compression” J. Biomech., 31, pp. 927-934;

[5] WO98/16644;

[6] WO2008/023063;

[7] WO2006/063362.

1-14. (canceled)

15. A method for short term storage of tissue comprising incubating or maintaining the tissue in a storage medium, wherein the storage medium comprise FGF-18.

16. The method according to claim 15, said method comprising incubating or maintaining the tissue in a storage medium at a temperature of about 20° C. to about 38° C. for up to 5 weeks and said method comprising:
a) incubating or maintaining the tissue for about 24 hours in fresh storage medium comprising FGF-18,
b) incubating or maintaining the tissue for about 144 hours in fresh storage medium which does not comprise FGF-18, and
c) repeating steps a) and b) until end of storage.

17. The method according to claim 15, wherein the storage medium is identical except for the presence of FGF-18.

18. The method according to claim 15, wherein the tissue is transplantation material of cartilage or osteochondral origin.

19. A method for long term storage of tissue comprising incubating or maintaining tissue in a storage medium for at least 5 weeks wherein the storage medium comprises FGF-18.

20. The method according to claim 19, said method comprising incubating or maintaining tissue in in a storage medium at a temperature of about 20° C. to about 38° C. for at least 5 weeks and said method comprising:
a) incubating or maintaining the tissue for about 24 hours in a fresh storage medium comprising FGF-18,
b) incubating or maintaining the tissue for about 144 hours in storage medium that does not comprise FGF-18, and
c) repeating steps a) and b) until end of storage.

21. A kit for preserving tissues including:
a) a storage medium comprising FGF-18, a vial for storage and instructions for use;
b) storage medium comprising FGF-18, a storage medium without FGF-18, a vial for storage and instructions for use;
c) a storage medium without FGF-18, separate FGF-18, a vial for storage and instructions for use; or
d) an FGF-18 compound, salts, amino acids, at least one preservative, at least one vitamin, a buffer and optionally a serum or a serum component.

22. The kit according to claim 21, said kit comprising storage medium comprising FGF-18, a storage medium without FGF-18, a vial for storage and instructions for use.

23. The kit according to claim 21, said kit comprising storage medium without FGF-18, separate FGF-18, a vial for storage and instructions for use.

24. The kit according to claim 21, said kit comprising storage medium comprises an FGF-18 compound, salts, amino acids, at least one preservative, at least one vitamin, a buffer and optionally a serum or a serum component.

25. The kit according to claim 21, wherein the FGF-18 is selected from the group consisting of:
a) a polypeptide comprising amino acid residues 28-207 of SEQ ID NO:1,
b) a polypeptide comprising amino acid residues 28-196 of SEQ ID NO:1, and
c) a polypeptide comprising SEQ ID NO:2.

26. The storage medium according to claim 24 comprising:
a) an FGF-18 compound comprising SEQ ID NO:2 at a concentration from about 10 to about 300 ng/mL,
b) penicillin at a concentration from about 10 U/mL to about 300 U/mL,
c) streptomycin at a concentration from about 10 U/mL to about 300 U/mL,
d) fungizone at a concentration from about 1 to about 10 μg/mL,
e) HEPES or HEPES/sodium bicarbonate buffer maintaining the medium at a pH between 7.2 and 7.4 at a concentration of 10 to 50 mM,
f) ascorbic acid at a concentration from about 10 to about 100 and μg/mL,
g) MEM vitamins, and
h) optionally a serum or a serum component.

27. The storage medium according to claim 26, said medium comprising a serum, said serum comprising fetal bovine serum at a concentration of 5 to 15%.

28. A method for preparing the storage medium comprising combining an FGF-18 compound, salts, amino acids, at least one preservative, at least one vitamin, a buffer and optionally a serum or a serum component to form said medium.
